(19) United States
(12) Patent Application Publication
(10) Pub. No.: US 2026/0100274 A1
(43) Pub. Date: Apr. 9, 2026

(54) MEDICAL DEVICE CONFIGURATION

(71) Applicant: Welch Allyn, Inc., Skaneateles Falls, NY (US)

(72) Inventors: Thomas A. Myers, Syracuse, NY (US); Deblina Pandit, Bangalore (IN)

(21) Appl. No.: 19/339,598

(22) Filed: Sep. 25, 2025

Related U.S. Application Data
(60) Provisional application No. 63/702,850, filed on Oct. 3, 2024.

(51) Int. Cl. G16H 40/40; A61B 5/00

(52) U.S. Cl. CPC G16H 40/40; A61B 5/746

(57) ABSTRACT

A system for configuring medical devices in a healthcare facility. The system receives a selection to configure one or more medical devices and generates a first electronic document that includes editable inputs for modifying one or more settings on the one or more medical devices. The system exports the first electronic document and receives one or more edits for modifying the one or more settings of the one or more medical devices and a signature. The system generates a second electronic document based on the one or more edits and exports the second electronic document for modifying the one or more settings of the one or more medical devices.

### CROSS-REFERENCE TO RELATED APPLICATION

This application claims the benefit of U.S. Provisional Application No. 63/702,850, filed October 3, 2024, the disclosure of which is hereby incorporated by reference in its entirety.

### BACKGROUND

Configuring medical device settings is crucial for ensuring accurate diagnostics and effective treatment. Typically, instructions for initial setup, calibration, and routine adjustments are included with each purchase of a medical device. The instructions guide users through setting up the medical device according to the manufacturer’s specifications, and typically include power management recommendations, optimal environmental conditions, and safety warnings.

When first setting up a medical device for the first time, users typically follow the manufacturer's instructions for assembly, connection to power sources, and initial software or firmware installation. This setup often includes configuring basic parameters like language preferences, date and time, and user profiles. Further, the medical device may also require calibration with standard reference materials to ensure accuracy.

After initial configuration, continuous monitoring is essential to ensure that the medical device functions correctly over time. Many medical devices come with diagnostic tools or alerts that notify users of any deviations or malfunctions. Regular maintenance, recalibration, and software updates are required to maintain the medical device’s performance. This ongoing process helps to ensure the medical device remains accurate and reliable throughout its use.

Medical devices are typically programmable, such that their operation can be tailored to specific needs of the environment where they are located. For instance, an infusion pump can be programmed to deliver a particular medication at a specific rate. The programming of medical devices should be done by trained professionals to avoid errors that could lead to patient harm.

### SUMMARY

In general terms, the present disclosure relates to medical device configuration. In one possible configuration, a first electronic document is used to generate a second electronic document for modifying one or more settings of one or more medical devices. Various aspects are described in this disclosure, which include, but are not limited to, the following aspects.

One aspect relates to a system for configuring medical devices in a healthcare facility, the system comprising: at least one processing device; and a memory device storing instructions which, when executed by the at least one processing device, cause the at least one processing device to: receive a selection to configure one or more medical devices; generate a first electronic document based on the selection to configure the one or more medical devices, the first electronic document including editable inputs for modifying one or more settings on the one or more medical devices; export the first electronic document to a workstation device; receive the first electronic document from the workstation device, the first electronic document including one or more edits for modifying the one or more settings of the one or more medical devices and a signature; generate a second electronic document based on the one or more edits in the first electronic document; and export the second electronic document for modifying the one or more settings of the one or more medical devices.

Another aspect relates to a method of configuring medical devices in a healthcare facility, the method comprising: receiving a selection to configure one or more medical devices; generating a first electronic document based on the selection to configure the one or more medical devices, the first electronic document including editable inputs for modifying one or more settings on the one or more medical devices; exporting the first electronic document; receiving the first electronic document with one or more edits for modifying the one or more settings on the one or more medical devices and a signature; generating a second electronic document based on the one or more edits in first electronic document; and exporting the second electronic document for modifying the one or more settings on the one or more medical devices.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for configuring medical devices in a healthcare facility.

FIG. 2 illustrates an example of a medical device included the system of FIG. 1.

FIG. 3 schematically illustrates an example of a workstation device that can be used to adjust one or more settings of the medical devices in the system of FIG. 1.

FIG. 4 schematically illustrates an example of a method of configuring one or more medical devices by the system of FIG. 1.

FIG. 5 schematically illustrates an example of a first electronic document that can be generated by the method of FIG. 4.

FIG. 6 illustrates component scores that are determined individually and summed together for calculating an early warning score in the first electronic document of FIG. 5.

FIG. 7 schematically illustrates an example of a configuration tool that can be used to convert a first electronic document into a second electronic document for modifying one or more settings of the medical devices in the system of FIG. 1.

### DETAILED DESCRIPTION

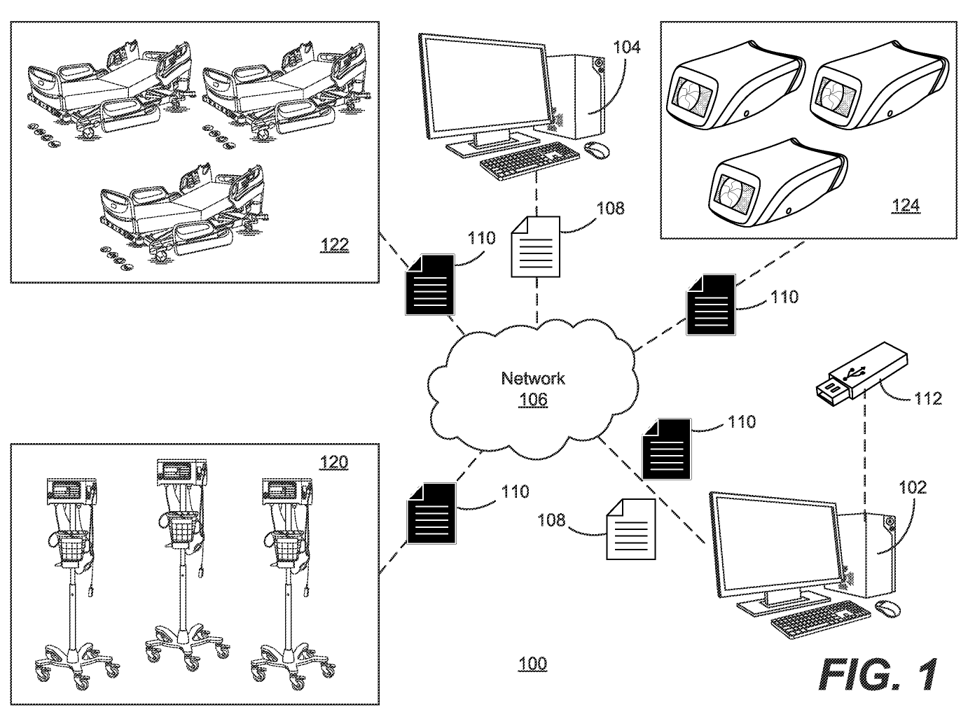

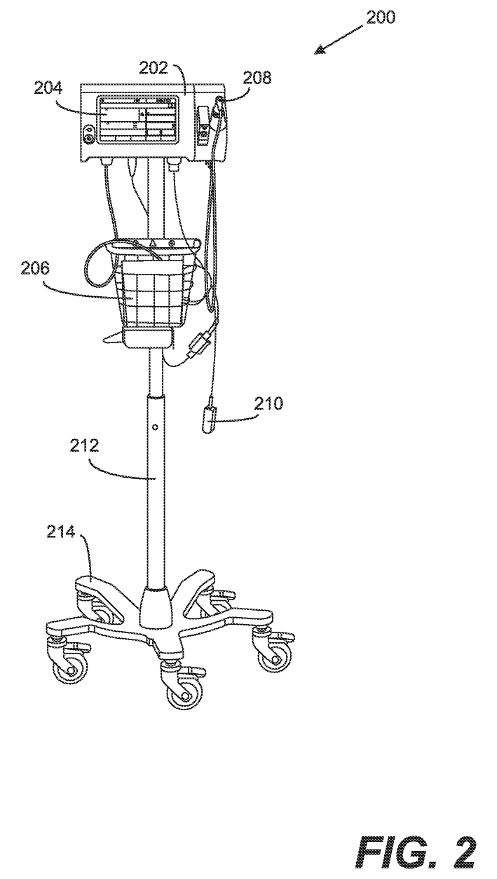

FIG. 1 schematically illustrates an example of a system 100 for configuring medical devices in a healthcare facility. As shown in FIG. 1, the system 100 can be used to configure various fleets of different types of medical devices. As used herein, a fleet of medical devices means a grouping or plurality of a certain type of medical device. For example, as shown in FIG. 1, a first fleet of medical devices 120 includes one or more patient monitoring devices, a second fleet of medical devices 122 includes one or more hospital beds, and a third fleet of medical devices 124 includes one or more vision screening and diagnostics devices. An example of a patient monitoring device is shown in FIG. 2, and is described in more detail further below.

FIG. 1 is provided by way of illustrative example such that the system 100 can be used to configure additional types of medical devices or fewer types of medical devices based on the needs of the healthcare facility where the system 100 is implemented. Further, each of the fleets can include one or more models or versions of a particular type of medical device. For example, the first fleet of medical devices 120 can include one or more models or versions of the patient monitoring devices. Similarly, the second fleet of medical devices 122 can include one or more models or versions of the hospital beds and the third fleet of medical devices 124 can include one or more models or versions of the vision screening and diagnostics devices.

The system 100 includes a first workstation device 102 operated by a first user who is trained to maintain the medical devices in the healthcare facility such as a biomedical equipment technician (“biomed”). The biomed can use the first workstation device 102 to perform tasks such as maintenance, repairs, procurement of new devices, and training of healthcare professionals (e.g., nurses, doctors, etc.) on how to use the medical devices. The first user is not authorized to change existing protocols, rules, and/or regulations regarding the operation and use of the medical devices in the healthcare facility where the system 100 is implemented.

The system 100 further includes a second workstation device 104 operated by a second user who is authorized to change existing protocols, rules, and/or regulations regarding the operation and use of the medical devices in the healthcare facility where the system 100 is implemented such as a member a hospital’s governing body or board of directors, a chief information officer (CIO), or other persons having similar roles. The second user is not trained to maintain the medical devices in the healthcare facility where the system 100 is implemented.

As shown in FIG. 1, the first workstation device 102 communicates with the second workstation device 104 over a network 106. The first workstation device 102 also communicates over the network 106 with the various fleets of medical devices such as the first fleet of medical devices 120, the second fleet of medical devices 122, and the third fleet of medical devices 124.

In some examples, the network 106 is a private network such as an intranet for sharing information, and providing communication and collaboration tools, and other services within the healthcare facility to the exclusion of access by outsiders. The first workstation device 102, the second workstation devices 104, and the fleets of medical devices can be connected over the network 106 by any types of wired or wireless connections or any combinations thereof, such as ethernet, Wi-Fi, and other communications technologies, mediums, and protocols.

As will be described in more detail, the first user of the first workstation device 102 generates a first electronic document 108 and communicates the first electronic document 108 to the second workstation device 104 over the network 106. The second user of the second workstation device 104 edits and signs the first electronic document 108, and returns over the network 106 the first electronic document 108 to the first workstation device 102. The first workstation device 102 then generates a second electronic document 110 based on the edits in the first electronic document 108, and exports the second electronic document 110 to adjust the configuration and settings of a fleet of one or more medical devices in the healthcare facility. In some examples, the second electronic document 110 is a design history file (DHF), a Device Master Record (DMR), or a Device History Record (DHR).

The first electronic document 108 has a first file format and the second electronic document 110 has a second file format. In some examples, the first file format is different from the second file format, as will be explained in more detail below.

The first file format of the first electronic document 108 is a standardized file type such as a portable document format (PDF) file format, a Word processing file format (e.g., .doc or .docx), or a spreadsheet file format (e.g., .xls or .xml). The first file format is used such that the second user of the second workstation device 104 is familiar with the file format such that they are able to edit the first electronic document 108 with minimal or no training.

The second file format of the second electronic document 110 is a configuration file that is recognized by the medical devices to update the configurations and settings of the medical devices. In some examples, the second file format of the second electronic document 110 is a JavaScript Object Notation (JSON) file format, an extensible markup language (XML) file format, or another type of file format. In some examples, the file format of the second electronic document 110 follows Fast Healthcare Interoperability Resources (FHIR), Health Level Seven (HL7), or other data standards for secure exchange of medical device communications.

As shown in FIG. 1, the second electronic document 110 can be exported by the first workstation device 102 to the medical devices over the network 106 to adjust the configurations and settings of the medical devices in the healthcare facility. Alternatively, the second electronic document 110 can be exported by the first workstation device 102 to a portable memory device 112 such as a thumb drive, a memory stick, a pen drive, and the like that can be physically inserted into each of the medical devices to adjust their configurations and settings.

FIG. 2 illustrates an example of a medical device 200 included the system 100. In this example, the medical device 200 is a patient monitoring device in the first fleet of medical devices 120. The medical device 200 includes a housing 202 having a display 204 that displays physiological parameters captured by one or more sensors that are integrated with or plugged into the housing 202 a such as a blood pressure sensor 206, a temperature sensor 208, and a pulse oximetry sensor 210. In some examples, the pulse oximetry sensor 210 includes a respiration rate sensor that can measure the respiration rate of a patient. In some examples, the display 204 is a touchscreen that receives tactile inputs from a user to enter information and to navigate between different tabs and/or pages within a software application displayed on the display 204.

As shown in FIG. 2, the housing 202, the display 204, and the one or more sensors are mounted on a portable frame 212 that includes casters 214 allowing the medical device 200 to be moved around the healthcare facility such as from patient room to patient room when being used for nurse rounding. In alternative examples, the medical device 200 can be stationary such that it can be mounted to a wall or attached to another fixture within the healthcare facility.

In examples where the medical device is a hospital bed, the medical device can include one or more sensors such as load sensors that detect the weight distribution and movements of a patient while resting on the hospital bed. The one or more sensors can further include a head section angle sensor that measures an angle of the head section of the hospital bed, a lift system position sensor that measures a height of the hospital bed, and one or more siderail position sensors that measure whether the siderails of the hospital bed are in the deployed position or in a stowed position. The load sensors can include one or more load cells, piezoelectric pressure sensors, strain gauges, and other types of sensors that can be positioned underneath a mattress where the patient rests to measure patient weight distribution and motion.

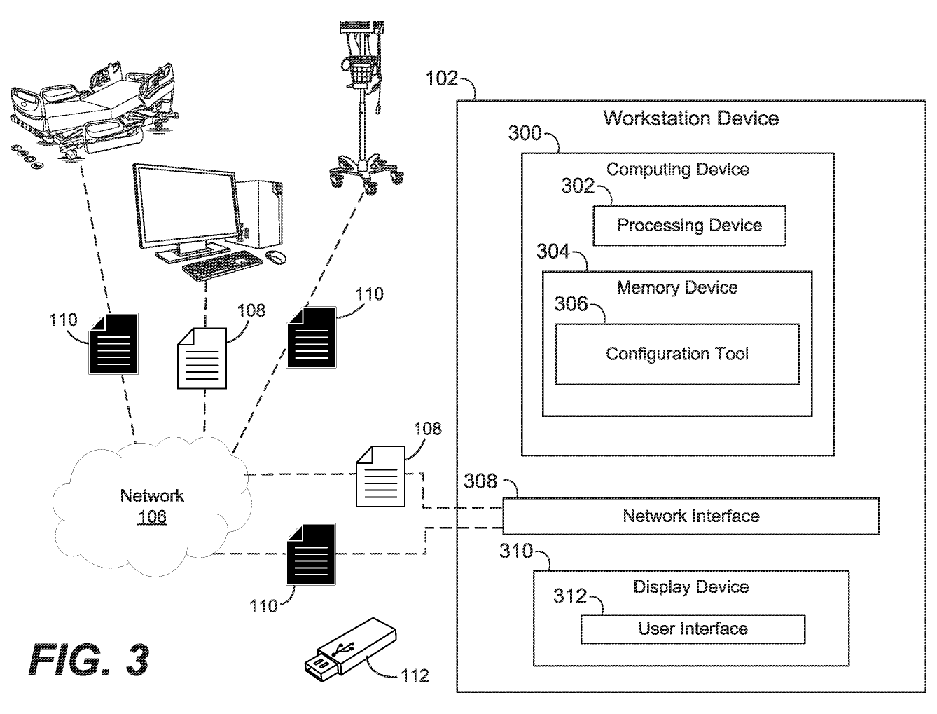

FIG. 3 schematically illustrates an example of the first workstation device 102 that can be used to adjust the configurations and the settings of the medical devices in the system 100. As shown in FIG. 3, the first workstation device 102 includes a computing device 300 having a processing device 302 and a memory device 304. The processing device 302 is an example of a processing unit such as a central processing unit (CPU). The processing device 302 can include one or more central processing units (CPUs). In some examples, the processing device 302 is part of a processing circuitry that can include one or more digital signal processors, field-programmable gate arrays, and other types of electronic circuits.

The memory device 304 operates to store data and instructions for execution by the processing device 302. As shown in FIG. 3, the memory device 304 stores a configuration tool 306 that can be used to update the configurations and settings of the medical devices in the healthcare facility. The configuration tool 306 will be described in more detail below.

The memory device 304 includes computer-readable media, which may include any media that can be accessed by the processing device 302. The computer-readable media includes non-transitory computer-readable storage media and computer-readable communication media.

The computer-readable storage media includes volatile and nonvolatile, removable and non-removable media implemented in any device configured to store information such as computer readable instructions, data structures, program modules, or other data. The computer-readable storage media can include, but is not limited to, random access memory, read only memory, electrically erasable programmable read only memory, flash memory, and other memory technology, including any medium that can be used to store information that can be accessed by the processing device 302. The computer-readable storage media is non-transitory.

The computer-readable communication media embodies computer-readable instructions, data structures, program modules, or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are within the scope of computer-readable media.

The first workstation device 102 includes a network interface 308 to connect the first workstation device 102 to the network 106. The connection to the network 106 allows the first workstation device 102 to communicate over the network 106 with the second workstation device 104 and with the fleets of medical devices in the healthcare facility such as the patient monitoring devices, the hospital beds, and the vision screening and diagnostics devices.

The network interface 308 can include a wired interface such as an ethernet cable port to connect the first workstation device 102 to the network 106, and can also include wireless interfaces to wirelessly connect the first workstation device 102 to the network 106 such as through Wi-Fi, ultra-wideband (UWB), and other types of wireless connections.

As described above, the first workstation device 102 generates a first electronic document 108 and communicates the first electronic document 108 to the second workstation device 104 over the network 106. The second workstation device 104 returns the first electronic document 108 with one or more edits and a signature to the first workstation device 102 over the network 106. The first workstation device 102 then generates a second electronic document 110 based on the one or more edits in the first electronic document 108, and then exports the second electronic document 110 to adjust one or more settings of one or more medical devices in the fleets of medical devices in the healthcare facility. In some examples, the first workstation device 102 can export the second electronic document 110 over the network 106 to the one or more medical devices. Alternatively, the first workstation device 102 can export the second electronic document 110 to a portable memory device 112 that can be physically inserted into each of the medical devices to adjust the one or more settings of the medical devices.

As further shown in FIG. 3, the first workstation device 102 includes a display device 310, which operates to display a user interface 312. In some examples, the display device 310 is a touchscreen such that the user interface 312 operates to receive inputs from the first user. In such examples, the display device 310 operates as both a display device and a user input device. The first workstation device 102 can also support peripheral devices such as a keyboard and mouse to receive inputs from the first user such as when using the configuration tool 306.

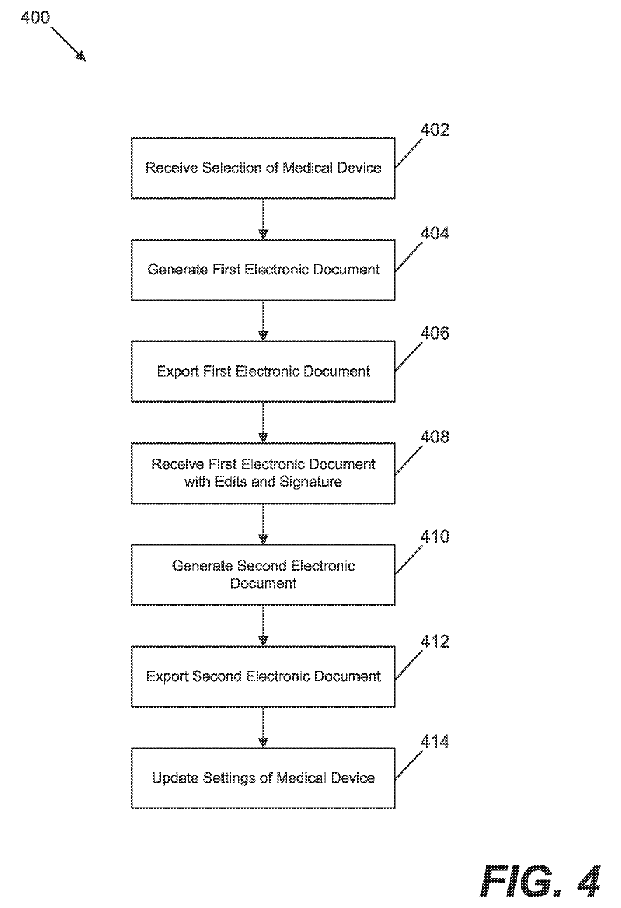

FIG. 4 schematically illustrates an example of a method 400 of configuring one or more medical devices by the system 100. The method 400 can be performed by the configuration tool 306 such as when installed or otherwise executed on the first workstation device 102.

The method 400 includes an operation 402 of receiving a selection of a type of medical device to adjust one or more settings of the one or more medical devices. Operation 402 can include receiving a selection of a patient monitoring device in the first fleet of medical devices 120. Alternatively, operation 402 can include receiving a selection of a hospital bed in the second fleet of medical devices 122. Alternatively, operation 402 can include receiving a selection of a vision screening and diagnostics device in the third fleet of medical devices 124. Alternatively, operation 402 can include receiving a selection of another type of medical device not shown in the examples provided in the figures. In some examples, the healthcare facility can include a plurality of medical devices of the type selected in operation 402.

The method 400 includes an operation 404 of generating the first electronic document 108 based on the selection received in operation 402. As described above, the first electronic document 108 has a standardized file type such as a portable document format (PDF) file format, a Word processing file format (e.g., .doc or .docx), a spreadsheet file format (e.g., .xls or .xml), or other standardized file type which the second user of the second workstation device 104 would be familiar with. As described above, the second user is not necessarily someone who is trained to use the configuration tool 306 installed on the first workstation device 102.

The first electronic document 108 generated in operation 404 includes editable inputs for modifying one or more settings of the selected one or more medical devices. The editable inputs are unique per type of medical device. For example, the editable inputs may differ depending on whether a hospital bed is selected in operation 402, or a vital signs monitoring device is selected in operation 402, or a vision screening device is selected in operation 402. The first electronic document 108 can be dynamically and/or periodically updated such as to include new settings or configurations for the one or more medical devices.

The method 400 includes an operation 406 of exporting the first electronic document 108 to the second workstation device 104. For example, operation 406 can include exporting the first electronic document 108 over the network 106 to the second workstation device 104. In some examples, the first electronic document 108 can be attached to electronic communications (e.g., email) sent to an account of the second user accessible on the second workstation device 104. In some examples, operation 406 can include exporting the first electronic document 108 to the portable memory device 112 that can be physically inserted into the second workstation device 104. In some examples, operation 406 includes printing a physical copy of the first electronic document 108 that can be sent to the second user for hand-written edits.

The method 400 includes an operation 408 of receiving the first electronic document 108 from the second workstation device 104 after the first electronic document 108 has been edited and signed by the second user. The first electronic document 108 includes one or more edits by the second user and a signature of the second user to confirm the identity of the second user. Operation 408 can include receiving the first electronic document 108 over the network 106 from the second workstation device 104. In some examples, operation 408 includes receiving the first electronic document 108 as an attachment in an electronic communication (e.g., an email) that is received by an account of the first user accessible on the first workstation device 102. In some examples, operation 408 includes receiving the first electronic document 108 on the portable memory device 112. In some examples, operation 408 includes receiving a printed copy of the first electronic document 108 with handwritten edits and signature.

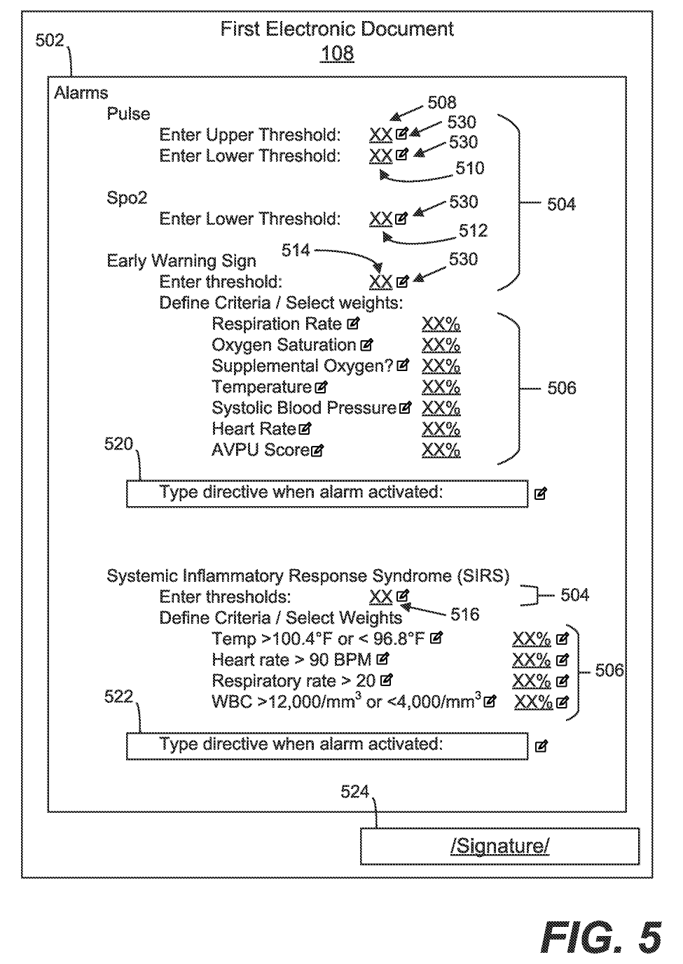

FIG. 5 schematically illustrates an example of the first electronic document 108 that can be generated, exported, and received in the method 400. The first electronic document 108 has a standardized file type such as a portable document format (PDF) file format, a Word processing file format (e.g., .doc or .docx), a spreadsheet file format (e.g., .xls or .xml), or other standardized file type that can be edited by the second user of the second workstation device 104.

The first electronic document 108 includes editable inputs 502 to adjust one or more settings on the one or more medical devices in the healthcare facility where the system 100 is implemented. The editable inputs 502 can include one or more thresholds 504 that can be adjusted for determining when to trigger alarms on the type of medical device selected in operation 402. For example, when the medical device 200 is selected in operation 402, the one or more thresholds 504 in the first electronic document 108 can include an upper threshold 508 that can be adjusted for determining when to trigger an alarm on the medical device 200 when a vital sign (e.g., pulse) measured by a sensor (e.g., the pulse oximetry sensor 210) is higher than the upper threshold 508. The one or more thresholds 504 can further include a lower threshold 510 that triggers the alarm on the medical device 200 when the vital sign (e.g., pulse) measured by the sensor (e.g., the pulse oximetry sensor 210) is lower than the lower threshold. The upper threshold 508 and the lower threshold 510 are editable as denoted by the edit symbol 530.

As another example, when the medical device 200 is selected in operation 402, the one or more thresholds 504 can include a lower threshold 512 that can be adjusted for determining when to trigger an alarm on the medical device 200 when a vital sign (e.g., SpO2) measured by a sensor (e.g., the pulse oximetry sensor 210) is lower than the lower threshold 512. The lower threshold 512 is editable as denoted by the edit symbol 530.

As a further example, the one or more thresholds 504 can include thresholds that determine when to trigger an alarm on the medical device 200 based on a composite score that is calculated based on physiological parameter measurements captured by the one or more sensors of the medical device 200. For example, a threshold 514 can be adjusted on the first electronic document 108 for determining when an alarm is triggered on the medical device 200 based on an early warning score (EWS). As another example, a threshold 516 can be adjusted on the first electronic document 108 for determining when an alarm is triggered on the medical device 200 based on a Systemic Inflammatory Response Syndrome (SIRS) for monitoring sepsis. The thresholds 514, 516 are editable as denoted by the edit symbol 530.

In view of the foregoing, the one or more thresholds 504 can be increased and/or decreased to adjust sensitivity on the selected type of medical device for triggering an alarm. For example, an upper threshold can be lowered and/or a lower threshold can be increased to increase the sensitivity on the medical device. Alternatively, the upper threshold can be increased and/or the lower threshold can be decreased to decrease the sensitivity on the medical device.

The editable inputs 502 on the first electronic document 108 can further include component scores 506 thar are used to compute a composite score (e.g., the EWS or SIRS) based on physiological parameter measurements captured by the one or more sensors of the medical device. The component scores 506 are editable on the first electronic document 108 for adjusting computation of the composite score on the medical device. The EWS is an example of a composite score that is calculated based on component scores 506 that include a respiration rate component score, a blood oxygen saturation component score, supplemental oxygen component score, a body temperature component score, a systolic blood pressure component score, a heart rate component score, and an Alert, Verbal, Pain, Unresponsive (AVPU) component score.

The SIRS is another example of a composite score that is calculated based on component scores 506 that include a body temperature component score, a heart rate component score, a respiration rate component score, and a white blood cell (WBC) component score. The first electronic document 108 can include editable inputs 502 for modifying the computation of additional composite scores, as well as for modifying the thresholds set for determining when to trigger an alarm on the selected type of medical device based on the additional composite scores.

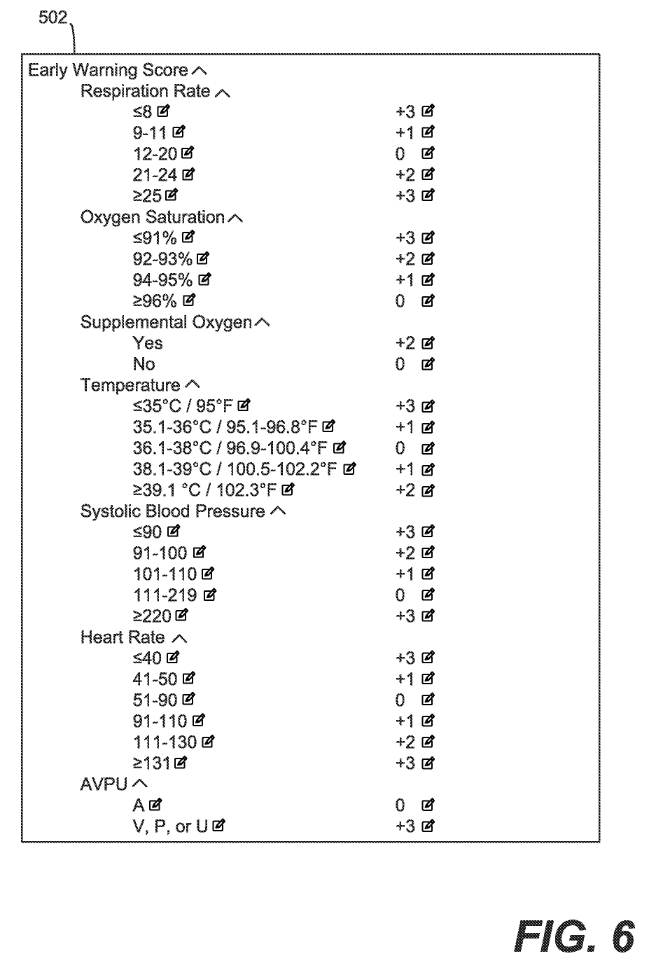

FIG. 6 illustrates the component scores that are determined individually and summed together for calculating the EWS shown in the example of the first electronic document 108 of FIG. 5. Each component score has one or more criteria. In some instances, the first electronic document 108 is designed to include a drop-down menu to expand the one or more criteria or to contract the one or more criteria for each component score of the EWS composite score. When expanded on the first electronic document 108, the second user is able to edit the criteria.

In the illustrative example provided in FIG. 6, the respiration rate component score has a value of +3 when the respiration rate is less than or equal to 8 breaths per minute (BPM), a value of +1 when the respiration rate is between 9-11 BPM, a value of 0 when the respiration rate is between 12-20 BPM, a value of +2 when the respiration rate is between 21-24 BPM, and a value of +3 when the respiration rate is more than or equal to 25 BPM.

The second user of the second workstation device 104 can edit any of the parameter ranges or score values on the first electronic document 108 for determining the respiration rate component score, as denoted by the edit symbol 530. For example, the second user can edit the respiration rate component score to have a value of +2 when the respiration rate is less than or equal to 8 BPM (instead of the original value of +3 shown in FIG. 6). As another example, the second user can define a broader range between 10 and 22 BPM for the value of 0 (instead of the original range of 12-20 BPM for the value of 0 shown in FIG. 6).

The blood oxygen saturation component score has a value of +3 when the blood oxygen saturation is less than or equal to 91%, a value of +2 when the blood oxygen saturation is between 92-93%, a value of +1 when the blood oxygen saturation is between 94-95%, a value of 0 when the blood oxygen saturation is more than or equal to 96%. Like in the example above, the second user of the second workstation device 104 can edit any of the parameter ranges or score values on the first electronic document 108 (as denoted by the edit symbols 530) for determining the blood oxygen saturation component score for calculating the EWS.

The supplemental oxygen component score has a value of +2 when the patient requires supplemental oxygen, and has a value of 0 when the patient does not require supplemental oxygen. The second user of the second workstation device 104 can edit the score values on the first electronic document 108 (see the edit symbols 530) for determining the supplemental oxygen component score for calculating the EWS composite score. For example, the second user can edit the supplemental oxygen component score to have a value of +3 when the patient requires supplemental oxygen (instead of the original value of +2 shown in FIG. 6).

The body temperature component score has a value of +3 when the body temperature is less than or equal to 95°F, a value of +1 when the body temperature is between 95.1-96.8°F, a value of 0 when the body temperature is between 96.9-100.4°F, a value of +1 when the body temperature is between 100.5-102.2°F, and a value of +2 when the body temperature is more than or equal to 102.3°F. Like in the examples above, the second user of the second workstation device 104 can edit any of the parameter ranges or score values (as denoted by the edit symbols 530) for determining the body temperature component score for calculating the EWS.

The systolic blood pressure component score has a value of +3 when the systolic blood pressure is less than or equal to 90 mm/Hg, a value of +2 when the systolic blood pressure is between 91-100 mm/Hg, a value of +1 when the systolic blood pressure is between 101-110 mm/Hg, a value of 0 when the systolic blood pressure is between 111-219 mm/Hg, and a value of +3 when the systolic blood pressure is more than or equal to 220 mm/Hg. Like in the examples above, the second user of the second workstation device 104 can edit any of the parameter ranges or score values on the first electronic document 108 (as denoted by the edit symbols 530) for determining the systolic blood pressure component score.

The heart rate component score has a value of +3 when the heart rate is less than or equal to 40 beats per minute (BPM), a value of +1 when the heart rate is between 41-50 BPM, a value of 0 when the heart rate is between 51-90 BPM, a value of +1 when the heart rate is between 91-110 BPM, and a value of +2 when the heart rate is between 111-130 BPM, and a value of +3 when the heart rate is more than or equal to 131 BPM. The second user of the second workstation device 104 can edit any of the parameter ranges or score values on the first electronic document 108 for determining the heart rate component score.

The component score based on AVPU has a value of 0 when the patient is alert and conscious (A), and has a value of +3 when the patient responds to verbal stimulus (V), responds to painful stimulus (P), or is unresponsive to any form of stimulus (U). The second user of the second workstation device 104 can edit the score values on the first electronic document 108 for determining the AVPU component score that is used for calculating the EWS composite score. For example, the second user can edit the AVPU component score to have a value of +4 when the patient is unresponsive to any form of stimulus (U), and to have a value of +2 when the patient responds to verbal stimulus (V) or responds to painful stimulus (P).

Referring back to FIG. 5, the editable inputs 502 in the first electronic document 108 further allow weights associated with any of the component scores to be edited for determining a composite score (e.g., EWS or SIRS). This is denoted in the first electronic document 108 by the edit symbols 530 next to percentages XX%. For example, the relative weights of the component scores can be adjusted such that one or more component scores are given more weight than one or more other component scores for calculating a composite score. In some instances, a weight for a particular component score can be set to zero such that the particular component score is given no weight (i.e., is ignored) when calculating a composite score.

As further shown in FIG. 5, the second user can similarly edit the parameter ranges, score values, and/or weights associated with the component scores of the SIRS composite score. The EWS and SIRS composite scores are provided for illustrative examples such that it is contemplated that the first electronic document 108 can allow the second user to edit the parameter ranges, score values, and/or weights of any kind of composite score that can trigger an alarm on the selected one or more medical devices in the healthcare facility.

As shown in FIG. 5, the first electronic document 108 includes areas 520, 522 where the second user can edit an existing directive or type a new directive for display on a display of the medical device when an alarm is triggered on the medical device. The area 520 allows entry of a new directive or editing of an existing directive for display on the medical device based the threshold 514 for the EWS. The area 522 allows entry of a new directive or editing of an existing directive for display on the medical device based the threshold 516 for the SIRS.

When the medical device 200 (i.e., the patient monitoring device) is selected in operation 402, the directives typed in the areas 520, 522 can be displayed on the display 204 of the medical device 200 when the thresholds 514, 516 are exceeded. As an illustrative example, a directive “Escalate patient status” can be typed or otherwise entered in the area 520 for display on the display 204 of the medical device 200 when the threshold 514 for the EWS is exceeded based on the physiological parameter measurements captured by the one or more sensors of the medical device 200. As another illustrative example, a directive “Order lactate lab analysis” can be typed or otherwise entered in the area 522 for display on the display 204 of the medical device 200 when the threshold 516 for the SIRS is exceeded based on the physiological parameter measurements captured by the one or more sensors of the medical device 200.

The directives typed in the areas 520, 522 can be based on a consensus reached by a governing body or board of directors on how to respond to clinical conditions of patients admitted to the healthcare facility where the system 100 is implemented. The system 100 allows new directives to be added or existing directives to be edited for display on the medical device.

As shown in FIG. 5, the first electronic document 108 further includes a signature area 524 where the second user enters an electronic signature once all of the edits on the first electronic document 108 are completed. Only users who have authority to change existing protocols, rules, and/or regulations regarding the operation and use of the medical devices in the healthcare facility where the system 100 is implemented are allowed to view, edit, sign, and return the first electronic document 108. In some examples, the electronic signature that is entered in the signature area 524 can be compared to a database of electronic signatures maintained by the system 100 to confirm the identity of the second user.

Referring back to FIG. 4, the method 400 includes an operation 410 of generating the second electronic document 110 based on the edits on the first electronic document 108 received in operation 408. The second electronic document 110 is a configuration file that can be read by the medical devices for updating one or more settings on the medical devices. For example, the second electronic document 110 can include a JavaScript Object Notation (JSON) file format, an extensible markup language (XML) file format, or another type of file format that is recognized by the one or more medical devices for adjusting the one or more settings of the devices.

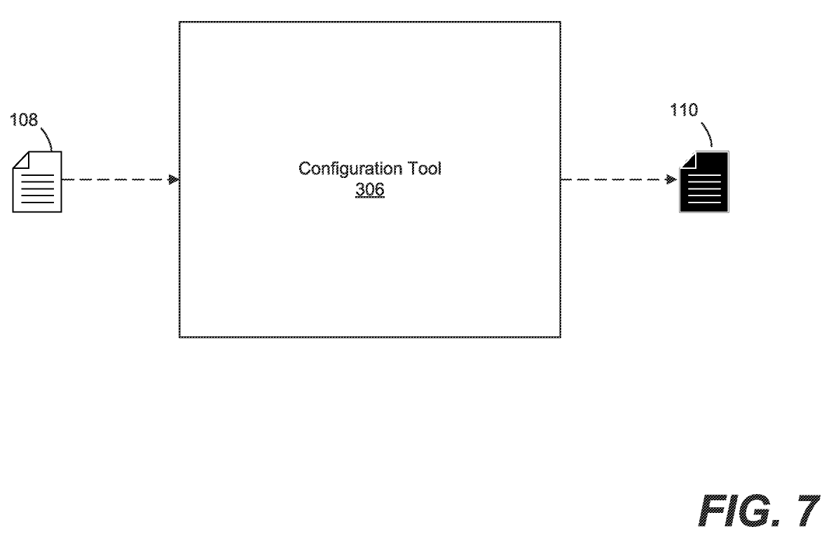

FIG. 7 schematically illustrates an example of the configuration tool 306 that can be used to convert the first electronic document 108 into the second electronic document 110 for modifying the one or more settings of the one or more medical devices in the system 100. As shown in FIG. 7, the configuration tool 306 receives the first electronic document 108 after it has been edited and signed by the second user.

The first electronic document 108 has the first file type such as a portable document format (PDF), a Word processing file format (e.g., .doc or .docx), a spreadsheet file format (e.g., .xls or .xml), or other standardized file types. The configuration tool 306 then converts the first electronic document 108 into the second electronic document 110 which has the second file format such as JavaScript Object Notation (JSON), extensible markup language (XML), or another type of file format that is recognized by the one or more medical devices.

Accordingly, after the first electronic document 108 is updated, signed, and returned, a new configuration file is created or an existing configuration file is updated by the configuration tool 306 with a unique versioning. The configuration tool 306 can employ one or more techniques for reading designated data fields of the first electronic document 108 to generate the new configuration file or to update the existing configuration file. The updates made to the first electronic document 108 are stored in a facility document management repository. The new configuration file is deployed and stored within the configuration tool 306.

Referring back to FIG. 4, once the second electronic document 110 is generated, the method 400 includes an operation 412 of exporting the second electronic document 110 for modifying the one or more settings of the one or more medical devices. In some examples, operation 412 includes exporting the second electronic document 110 over the network 106 directly to the medical devices. Alternatively, operation 414 can include exporting the second electronic document 110 to a portable memory device 112 such as a thumb drive, a memory stick, a pen drive, and the like that can be physically inserted into each of the medical devices.

The method 400 includes an operation 414 of updating the one or more settings of the one or more medical devices based on the second electronic document 110. For example, when exported to the medical device, the second electronic document 110 is recognized by the medical device as a configuration file. In some instances, medical device automatically downloads the configuration file onto a memory of the medical device, which causes an automatic adjustment of the one or more settings on the medical device. Alternatively, the medical device can request confirmation from an authorized user before the medical device downloads the configuration file onto the memory for adjustment of the one or more settings on the medical device. Whether the configuration file is automatically downloaded or confirmation is first requested before the downloading can depend on the protocols of the facility where the medical device is deployed.

The various embodiments described above are provided by way of illustration only and should not be construed to be limiting in any way. Various modifications can be made to the embodiments described above without departing from the true spirit and scope of the disclosure.

What is claimed is:

1. A system for configuring medical devices in a healthcare facility, the system comprising:
at least one processing device; and
a memory device storing instructions which, when executed by the at least one processing device, cause the at least one processing device to:
receive a selection to configure one or more medical devices;
generate a first electronic document based on the selection to configure the one or more medical devices, the first electronic document including editable inputs for modifying one or more settings on the one or more medical devices;
export the first electronic document to a workstation device;
receive the first electronic document from the workstation device, the first electronic document including one or more edits for modifying the one or more settings of the one or more medical devices and a signature;
generate a second electronic document based on the one or more edits in the first electronic document; and
export the second electronic document for modifying the one or more settings of the one or more medical devices.

2. The system of claim 1, further comprising:
the one or more medical devices, and wherein the one or more medical devices each include at least one sensor for measuring a physiological parameter of a patient.

3. The system of claim 2, wherein the at least one sensor includes a blood pressure sensor, a temperature sensor, a pulse oximetry sensor, or a respiration rate sensor.

4. The system of claim 1, wherein the editable inputs in the first electronic document are based on a type of the one or more medical devices, and the types of the one or more medical devices include hospital beds, vital signs monitoring devices, and vision screening devices.

5. The system of claim 1, wherein the one or more edits in the first electronic document cause the second electronic document to adjust computation of a composite score based on physiological parameters measured by the one or more medical devices.

6. The system of claim 1, wherein the one or more edits in the first electronic document cause the second electronic document to modify a directive displayed on the one or more medical devices.

7. The system of claim 1, wherein the one or more edits in the first electronic document cause the second electronic document to adjust one or more thresholds for triggering alarms on the one or more medical devices.

8. The system of claim 7, wherein the one or more thresholds include a threshold that triggers an alarm on the one or more medical devices when a composite score exceeds the threshold.

9. The system of claim 1, wherein the second electronic document is exported over a network to the one or more medical devices.

10. The system of claim 1, wherein the second electronic document is exported to a portable memory device.

11. A method of configuring medical devices in a healthcare facility, the method comprising:
receiving a selection to configure one or more medical devices;
generating a first electronic document based on the selection to configure the one or more medical devices, the first electronic document including editable inputs for modifying one or more settings on the one or more medical devices;
exporting the first electronic document;
receiving the first electronic document with one or more edits for modifying the one or more settings on the one or more medical devices and a signature;
generating a second electronic document based on the one or more edits in first electronic document; and
exporting the second electronic document for modifying the one or more settings on the one or more medical devices.

12. The method of claim 11, wherein the editable inputs in the first electronic document are unique per type of medical device, and the types of medical devices include one or more hospital beds, one or more vital signs monitoring devices, and one or more vision screening devices.

13. The method of claim 11, wherein the one or more edits in the first electronic document cause the second electronic document to adjust computation of a composite score based on physiological parameters measured by at least one sensor.

14. The method of claim 11, wherein the one or more edits in the first electronic document cause the second electronic document to modify a directive for display on the one or more medical devices.

15. The method of claim 11, wherein the one or more edits in the first electronic document cause the second electronic document to adjust one or more thresholds for triggering alarms on the one or more medical devices.

16. The method of claim 15, wherein the one or more thresholds include an upper threshold and a lower threshold that trigger the alarm on the one or more medical devices when a physiological parameter measured by at least one sensor of the one or more medical devices is above the upper threshold or lower than the lower threshold.

17. The method of claim 16, wherein the at least one sensor includes a blood pressure sensor, a temperature sensor, a pulse oximetry sensor, or a respiration rate sensor.

18. The method of claim 15, wherein the one or more thresholds include a threshold that triggers an alarm on the one or more medical devices when a composite score exceeds the threshold.

19. The method of claim 11, wherein the second electronic document is exported over a network to the one or more medical devices.

20. The method of claim 11, wherein the second electronic document is exported to a portable memory device.
